(19) United States
(12) Patent Application Publication
(10) Pub. No.: US 2024/0260829 A1
(43) Pub. Date: Aug. 8, 2024

(54) EXERCISE PERFORMANCE ESTIMATION APPARATUS, EXERCISE PERFORMANCE ESTIMATION METHOD, AND PROGRAM

(71) Applicant: NIPPON TELEGRAPH AND TELEPHONE CORPORATION, Tokyo (JP)

(72) Inventor: Naoki SAIJO, Tokyo (JP)

(73) Assignee: NIPPON TELEGRAPH AND TELEPHONE CORPORATION, Tokyo (JP)

(21) Appl. No.: 18/562,779

(22) PCT Filed: May 26, 2021
(86) PCT No.: PCT/JP2021/019977

§ 371 Date: Nov. 20, 2023

(51) Int. Cl. A61B 3/113

(52) U.S. Cl. CPC A61B 3/113

(57) ABSTRACT

A motion performance estimation device obtains and outputs an index representing motion performance of a subject on the basis of a difference in a feature amount based on a microsaccade between the subject performing a task according to movement of a first object and the subject performing a task according to movement of a second object. However, the size of the visual angle formed by the first object and the size of the visual angle formed by the second object in the eye of the subject are different.

### TECHNICAL FIELD

The present invention relates to a technology of estimating motion performance (motion characteristics) of a subject.

### BACKGROUND ART

There is known a technology of estimating a size of an attention range (observation range) from movement of the eye of a subject during motion and estimating motion performance of the subject on the basis of the size of the attention range using a property that information of a microsaccade of the eye of the subject is correlated with the size of the attention range of the subject and a property that the size of the attention range is correlated with a reaction speed or accuracy of a reaction of the subject (see, for example, Patent Literature 1).

### CITATION LIST

### Patent Literature

Patent Literature 1: JP 2019-30491 A

### SUMMARY OF INVENTION

### Technical Problem

The technology of Patent Literature 1 evaluates motion performance of a subject using minute movement of the eyeball unconsciously generated when the subject gazes at an object. On the other hand, ability to appropriately adjust an attention range according to the surrounding situation is required in actual motion performance, and evaluating the motion performance only on the basis of minute movement of the eyeball without considering the surrounding situation is difficult.

The present invention has been made in view of such a point, and an object thereof is to provide a technology of appropriately evaluating motion performance according to a surrounding situation.

### Solution to Problem

A motion performance estimation device obtains and outputs an index representing motion performance of a subject on the basis of a difference in a feature amount based on a microsaccade between the subject performing a task according to movement of a first object and the subject performing a task according to movement of a second object. However, the size of the visual angle formed by the first object and the size of the visual angle formed by the second object in the eye of the subject are different.

### Advantageous Effects of Invention

As a result, motion performance according to a surrounding situation can be appropriately evaluated.

### BRIEF DESCRIPTION OF DRAWINGS

FIG. 1 is a block diagram illustrating a functional configuration of a motion performance estimation system according to a first embodiment.

FIG. 2A is a diagram illustrating a video (Wide-view) in which a small object is displayed, and FIG. 2B is a diagram illustrating a video (Zoomed-view) in which a large object is displayed. FIGS. 2C to 2F are graphs each illustrating relationship between the size of an object and a feature amount of microsaccades of a skilled person (Skilled) and an unskilled person (Sub-skilled).

FIGS. 3A and 3B are graphs each illustrating relationship between an attention range and a feature amount of a microsaccade.

FIG. 4 is a block diagram illustrating a functional configuration of a motion performance estimation system according to a second embodiment.

FIG. 5 is a block diagram illustrating a hardware configuration of a motion performance estimation device.

### DESCRIPTION OF EMBODIMENTS

Hereinafter, embodiments of the present invention will be described with reference to the drawings.

### [Principles]

First, experimental results as a premise of the present invention will be described.

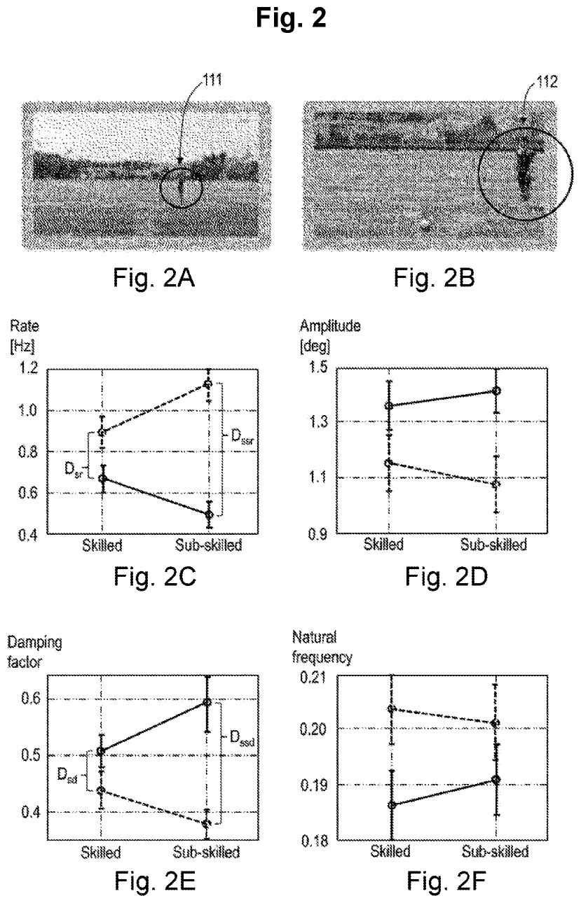

In this experiment, a testee (subject) is caused to view a video for about 2 seconds that is a scene of a penalty kick viewed from a goalkeeper's viewpoint of soccer and a moment at which a kicker 111, 112 (object) runs from a position rightward from the center of the screen toward a ball in the center of the screen and kicks the ball, and is caused to predict whether the ball will fly right or left at the next time, that is, whether the kicker 111, 112 will kick the ball right or left (FIGS. 2A and 2B). Such a task according to the movement of an object is referred to as a “prediction task”.

At this time, eye movement of the testee viewing the video is acquired by an eyeball motion measurement device such as an eye tracker. Videos in which the kickers 111, 112 have different sizes in the videos on the screen are prepared, and the same prediction task is performed on each of the videos. Note that the difference in the sizes of the kickers 111, 112 in the videos means that the size of the visual angle formed by the kicker 111 in the video and the size of the visual angle formed by the kicker 112 in the eye of the testee are different. Note that the size of the visual angle formed by the object in the video (for example, kicker 111, 112 in the video) in the eye of the testee may be the size of the visual angle in the vertical direction of the object in the video in the eye of the testee (for example, size of the visual angle of a region from the tip of the foot to the tip of the head of the kicker 111, 112 in the video), the size of the visual angle in the horizontal direction of the object in the video in the eye of the testee (for example, size of the visual angle of a region between both shoulders of the kicker 111, 11 in the video), or the size of the visual angle in other directions of the object in the video in the eye of the testee. In the examples of FIGS. 2A and 2B, the size of the kicker 111 in the video is smaller than the size of the kicker 112, and the size of the visual angle formed by the kicker 111 in the video in the eye of the testee is smaller than the size of the visual angle formed by the kicker 112.

The start time and the size of jumping eyeball motion (saccade) are obtained using information such as the direction, the angular velocity, and the angular acceleration of the eyeball for each time acquired by the eyeball motion measurement device. The saccade includes minute jumping eyeball motion (microsaccade) that has an amplitude of about 1° and occurs only unconsciously, and jumping eyeball motion that has a larger amplitude than the minute jumping eyeball motion and is caused to also occur consciously. Here, the former is targeted. That is, eyeball motion in which the maximum angular velocity and the maximum angular acceleration are within predetermined reference values is detected as a microsaccade from movement of the eyeball at each time acquired by the eyeball motion measurement device. Next, feature amounts based on a microsaccade including the occurrence frequency (Rate) and the amplitude (Amplitude) of a microsaccade of the testee when the testee is viewing the scene (hereinafter, the occurrence frequency may be simply referred to as occurrence frequency) (hereinafter, the amplitude may be simply referred to as amplitude), and an attenuation coefficient (Damping factor) and the natural frequency (Natural frequency) when the eye of the testee is modeled by dynamics of a secondary system (hereinafter, the attenuation coefficient may be simply referred to as attenuation coefficient) (hereinafter, the natural frequency may be simply referred to as natural frequency) (hereinafter, the feature amounts may be simply referred to as “feature amounts”) are calculated, and the average values of the respective feature amounts are obtained. Note that the natural frequency is synonymous with a unique frequency (also referred to as unique vibration frequency), and a natural frequency f and a unique angular vibration frequency ω satisfy relationship of ω=2πf. The motion performance of the testee can be predicted by whether these feature amounts based on a microsaccade are appropriately adjusted according to the size of the kicker 111, 112 in the video on the screen.

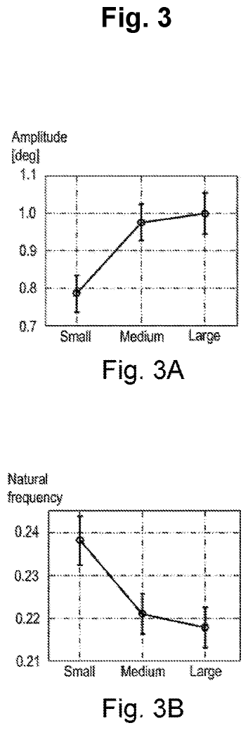

FIGS. 2C to 2F each illustrate relationship between the size of the kicker 111, 112 (object) in the video that testees are viewing (size of the visual angle formed by the kicker 111, 112 in the video in the eyes of the testees. Hereinafter, the size of the visual angle formed by the kicker in the video in the eye of a testee is described as the size of the kicker, the fact that the visual angle formed by the kicker in the video in the eye of a testee is large is described as the kicker is large, the kicker in which the visual angle formed by the kicker in the video in the eye of a testee is large is described as a large kicker, and the kicker in which the visual angle formed by the kicker in the video in the eye of a testee is small is described as a small kicker), and a feature amount based on a microsaccade in a case of a person skilled in playing soccer (hereinafter, referred to as skilled person [Skilled]) and in a case of a person other than a skilled person (hereinafter, referred to as unskilled person [Sub-skilled]). FIGS. 2C to 2F illustrate data obtained assuming a player of a first string of a soccer team as a skilled person and a player of a second or lower string of the same team as an unskilled person. The horizontal axes in FIGS. 2C to 2F each represent whether the testee is a skilled person or an unskilled person. The vertical axis in FIG. 2C represents the occurrence frequency (Rate) [Hz], the vertical axis in FIG. 2D represents the amplitude (Amplitude) [deg], the vertical axis in FIG. 2E represents the attenuation coefficient (Damping factor), and the vertical axis in FIG. 2F represents the natural frequency (Natural frequency). Broken lines in FIGS. 2C to 2F each represent a result in a case where the prediction task is performed on a video of the small kicker 111 (Wide-view in FIG. 2A), and solid lines each represent a result in a case where the prediction task is performed on a video of the large kicker 112 (Zoomed-view in FIG. 2B). Circles in FIGS. 2C to 2F each represent the average values of the respective feature amounts. Furthermore, FIGS. 3A and 3B each illustrate relationship between an attention range that is a range to which the testee pays attention (also referred to as “observation range”) and feature amounts based on a microsaccade of the eye of the testee. A method of measuring the attention range is disclosed in Patent Literature 1. The horizontal axes in FIGS. 3A and 3B each represent the attention range. Here, three categories of “Large”, “Medium”, and “Small” are adopted as the attention range. The vertical axis in FIG. 3A represents the amplitude (Amplitude) [deg], and the vertical axis in FIG. 3B represents the natural frequency (Natural frequency). A skilled person and an unskilled person are caused to view videos on which the kickers 111, 112 having different sizes are displayed as described above, and change in feature amounts based on a microsaccade when the prediction task is performed is analyzed using these graphs.

As illustrated in FIGS. 2D, 2F, 3A, and 3B, both the skilled person and the unskilled person have a tendency that the amplitude increases and the natural frequency decreases as the kicker that is the object viewed by the testees is larger, which indicates that the attention range is widened according to the size of the kicker. On the other hand, the difference between the skilled person and the unskilled person is remarkably represented in the occurrence frequency and the attenuation coefficient. As illustrated in FIGS. 2C and 2E, in a case of the small kicker 111 (FIG. 2A: Wide-view), the occurrence frequency of a microsaccade is low and the attenuation coefficient is large in the skilled person (Skilled) as compared with the unskilled person (Sub-skilled). This indicates that the field of view of the skilled person is narrower than that of the unskilled person. The large kicker 112 (FIG. 2B: Zoomed-view), the occurrence frequency of a microsaccade is high and the attenuation coefficient is small in the skilled person (Skilled) as compared with the unskilled person (Sub-skilled). This indicates that the field of view of the skilled person is wider than that of the unskilled person. That is, as compared with the unskilled person, the difference in the occurrence frequency and the attenuation coefficient of a microsaccade is smaller between a case where the skilled person is performing the prediction task by viewing the small kicker 111 (hereinafter referred to as “prediction task A”) and a case where the skilled person is performing the prediction task by viewing the large kicker 112 (hereinafter referred to as “prediction task B”). That is, there is a tendency that relationship of Dₛᵣ<Dₛₛᵣ is satisfied, where Dₛᵣ is the difference in the occurrence frequency of a microsaccade between a case where the skilled person is performing the prediction task A and a case where the skilled person is performing the prediction task B, and Dₛₛᵣ is the difference in the occurrence frequency of a microsaccade between a case where the unskilled person is performing the prediction task A and a case where the unskilled person is performing the prediction task B (FIG. 2C). Similarly, there is a tendency that relationship of D_{sd}<D_{ssd} is satisfied, where D_{sd} is the difference in the attenuation coefficient of a microsaccade between a case where the skilled person is performing the prediction task A and a case where the skilled person is performing the prediction task B, and D_{ssd} is the difference in the attenuation coefficient of a microsaccade between a case where the unskilled person is performing the prediction task A and a case where the unskilled person is performing the prediction task B (FIG. 2E). The tendency that the magnitude of the difference between a feature amount based on a microsaccade in a case where the prediction task A is performed and the feature amount based on a microsaccade in a case where the prediction task B is performed is different between the skilled person and the unskilled person is also observed in the amplitude, the natural frequency, and the like (FIGS. 2D and 2F). However, the tendency is more remarkable in the occurrence frequency and the attenuation coefficient of a microsaccade.

On the basis of the above finding, in each embodiment, the correlation between the difference in a feature amount based on a microsaccade of the eye of a subject according to the size of the visual angle formed by an object in the eye of the subject (size of the object) and the degree of motion proficiency of the subject is used, and motion performance of the subject (degree of proficiency or the presence or absence of potential prediction ability) is estimated from a feature amount based on a microsaccade of the eye of the subject performing a task according to the movement of the object.

### First Embodiment

A first embodiment will be described.

### <Configuration>

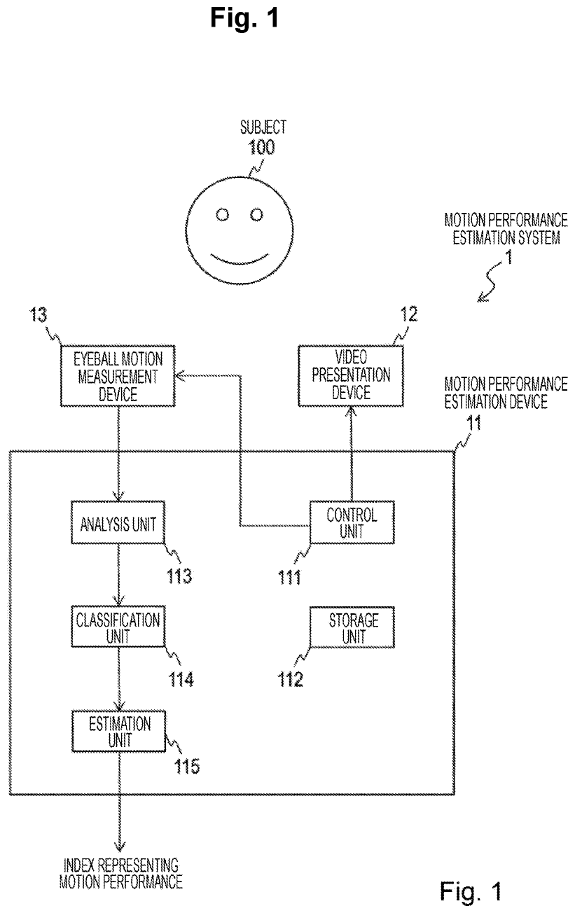

As illustrated in FIG. 1, a motion performance estimation system 1 according to the present embodiment includes a motion performance estimation device 11 that estimates motion performance of a subject 100, a video presentation device 12 that presents (displays) a video including an object, and an eyeball motion measurement device 13 that measures eyeball motion of the subject 100. The motion performance estimation device 11 includes a control unit 111, a storage unit 112, an analysis unit 113, a classification unit 114, and an estimation unit 115. The motion performance estimation device 11 performs each type of processing on the basis of the control unit 111, and data obtained in each processing is stored in the storage unit 112 one by one, and read and used as necessary. The video presentation device 12 is a device such as a display or a projector that presents a video including an object. The eyeball motion measurement device 13 is a device such as an eye tracker that measures eyeball motion of the subject 100.

### <Processing>

The motion performance estimation device 11 obtains and outputs an index representing motion performance of the subject 100 on the basis of the difference in a feature amount based on a microsaccade between the subject 100 performing a task according to movement of a first object in a video presented from the video presentation device 12 (hereinafter, simply referred to as in the video) and the subject 100 performing a task according to movement of a second object in the video. However, the size of the visual angle formed by the first object in the video and the size of the visual angle formed by the second object in the video in the eye of the subject 100 are different. Hereinafter, an example of this processing will be described.

The control unit 111 selects one measurement condition from a plurality of measurement conditions prepared in advance. The measurement condition is a condition of an object corresponding to a task to be performed by the subject 100 (for example, task of predicting a result brought by motion of the object), and includes a condition of the size of the object in the video (size of the visual angle formed by the object in the eye of the subject 100) and a condition of a result according to the movement of the object in the video. Since the subject 100 performs a task according to the movement of the object, the measurement condition can also be said to be information for identifying the task of the subject 100. For example, in a case where the subject 100 is caused to perform a task of predicting whether the ball kicked by the kicker (object) flies to the right or the left in the scene of the penalty kick described above, conditions of the size of the object in the video are “the kicker is imaged small in the video (Wide-view)” and “the kicker is imaged large in the video (Zoomed-view)”, and conditions of the result according to the movement of the object are “the ball flies to the right” and “the ball flies to the left”, and thus four types of measurement conditions of combinations thereof are prepared in advance. Furthermore, for example, in a case where the subject 100 is caused to perform a task of predicting a movement direction of an opponent (object) who approaches holding a ball in a scene such as rugby, conditions of the size of the object in the video are “the opponent is imaged small in the video“and” the opponent is imaged large in the video”, and conditions of the result according to the movement of the object are “the opponent moves right” and “the opponent moves left”, and thus four types of measurement conditions of combinations thereof are prepared in advance. The control unit 111 may randomly select a measurement condition, may select a measurement condition on the basis of input from the outside, or may select a measurement condition according to a predetermined order (step S111).

The control unit 111 performs control to cause the video presentation unit 12 to present a video indicating movement of the object corresponding to the selected measurement condition. The video presentation unit 12 receives information of the control from control unit 111 as input, and outputs the designated video. The video presentation unit 12 presents (displays) the designated video to the subject 100 under the control of the control unit 111. The video is a video including movement of the object having the size indicated by the selected measurement condition, and is a video for causing prediction of a result according to the movement of the object indicated by the measurement condition. For example, this video is a video obtained by performing capturing from the viewpoint of a person who acts according to the movement of the object in a predetermined time section, includes the movement of the object having a size indicated by the selected measurement condition, and is a video from which the result according to the movement of the object indicated by the measurement condition at a time next to the predetermined time section is predicted. In other words, this video is, for example, a video indicating movement performed by the object having a size represented by the selected measurement condition immediately before the result indicated by the selected measurement condition. For example, in the former example, in a case where the selected measurement condition is a combination of “the kicker is imaged small in the video” and “the ball flies to the right”, the video presentation unit 12 extracts and presents a video from the beginning of a video to a moment when the kicker runs from the position closer to the right than the center of the screen toward the ball in the center of the screen and kicks the ball from the video of a scene where the kicker imaged small in the video runs from the position closer to the right than the center of the screen toward the ball in the center of the screen and kicks the ball, and the ball flies to the right. Furthermore, in the latter example, in a case where the selected measurement condition is a combination of “the opponent is imaged large in the video” and “the opponent moves to the left”, the video presentation unit 12 presents a video from the beginning through a scene where the opponent comes to the camera holding the ball to immediately before the opponent moves to the left in front of the camera from a video of a scene where the opponent imaged large in the video comes to the camera holding the ball and moves to the left in front of the camera, that is, in front of the eye of the video viewer (step S12).

Furthermore, the control unit 111 transmits the selected measurement condition to the eyeball motion measurement device 13, and controls the eyeball motion measurement device 13 to acquire eyeball motion of the subject 100 while the video presentation unit 12 presents a video corresponding to the measurement condition. Thus, the eyeball motion measurement device 13 measures the eyeball motion (for example, the position of the eyeball at each time point) of the subject 100 to which the video corresponding to the measurement condition is presented. Note that, in the present embodiment, regardless of the measurement condition, the distance between the video presentation unit 12 that presents the video corresponding to the measurement condition and the subject 100 is constant or substantially constant. The measurement result of the eyeball motion is output to the analysis unit 113 in association with the measurement condition (step S13).

The measurement result of the eyeball motion and the measurement condition associated with the measurement result are input to the analysis unit 113. The analysis unit 113 extracts feature amounts based on a microsaccade of the eye of the subject 100 from the input measurement result of the eyeball motion (for example, time-series information of the position of the eyeball). For example, the analysis unit 113 calculates the maximum angular velocity or the maximum angular acceleration of the eyeball motion from the time-series information of the position of the eyeball, extracts time-series information of the time when the result exceeds a predetermined reference value (time when a microsaccade is generated) and the amplitude thereof (magnitude of the microsaccade), and extracts feature amounts based on a microsaccade from the time-series information. Examples of the feature amounts based on a microsaccade include the following.
(1) Feature amount representing the occurrence frequency of a microsaccade
(2) Feature amount representing the amplitude of a microsaccade
(3) Feature amount representing the attenuation coefficient of a microsaccade when the eye is modeled by dynamics of the secondary system
(4) Feature amount representing the unique angular vibration frequency of a microsaccade when the eye is modeled by dynamics of the secondary system

The feature amount (1) may be the occurrence frequency of a microsaccade or a function value of the occurrence frequency. The feature amount (2) may be the amplitude of a microsaccade or a function value of the amplitude (for example, power). The feature amount (3) may be the attenuation coefficient of a microsaccade or a function value of the attenuation coefficient. The feature amount (4) may be the unique angular vibration frequency of a microsaccade or a function value of the unique angular vibration frequency (for example, natural frequency). Note that while the feature amount (1) is obtained for each predetermined time section belonging to a time section in which the video corresponding to the measurement condition input to analysis unit 113 is presented from video presentation unit 12 (hereinafter, the time section is referred to as a presentation time section) (the predetermined time section is, for example, a time section or a time frame of one see or more immediately before the end time of the video presented from the video presentation device 12), the feature amounts (2) to (4) can also be obtained for each time belonging to the presentation time section. In a case of a time belonging to the presentation time section, at least one of the feature amounts (2) to (4) may be obtained at a time belonging to a time section in which the feature amount (1) is obtained, or at least one of the feature amounts (2) to (4) may be obtained at a time different from the time section in which the feature amount (1) is obtained. Furthermore, each of the feature amounts may be extracted one by one from the measurement result of the eyeball motion, or may be a function value of a plurality of values (for example, average value or representative value) extracted from the measurement result of the eyeball motion. The analysis unit 113 may extract all the feature amounts (1) to (4), or may extract only a part of the feature amounts. For example, the analysis unit 113 may extract the feature amount of (1) or (3) (first feature amount representing the occurrence frequency or the attenuation coefficient of a microsaccade), or may extract the feature amount of (1) or (3) and the feature amount of (2) or (4) (second feature amount representing the amplitude or the unique angular vibration frequency of a microsaccade). The analysis unit 113 outputs the extracted feature amount based on a microsaccade and the measurement condition associated with the measurement result of the eyeball motion that is the basis of the feature amount to the classification unit 114 in association with each other (step S113).

Processing of steps S111, S12, S13, and S113 described above is performed a plurality of times while the measurement condition is changed. As a result, at least feature amounts based on a plurality of microsaccades of the subject 100 are obtained for objects having different sizes in videos. That is, at least a feature amount based on a microsaccade of the subject 100 performing a task according to movement of a first object in a video presented from the video presentation device 12 and a feature amount based on a microsaccade of the subject 100 performing a task according to movement of a second object in a video presented from the video presentation device 12 are obtained. However, sizes of the first object and the second object in the videos are different from each other. In other word, the size of the visual angle formed by the first object in the video and the size of the visual angle formed by the second object in the video in the eye of the subject 100 are different from each other.

A plurality of measurement conditions and feature amounts based on a microsaccade associated with the plurality of measurement conditions are input to the classification unit 114. The classification unit 114 classifies the feature amounts based on a microsaccade for each of the measurement conditions, and collectively outputs the feature amounts based on a microsaccade corresponding to the measurement conditions to the estimation unit 115 for each of the measurement conditions. For example, the classification unit 114 integrates and outputs feature amounts based on a microsaccade corresponding to the same measurement condition. For example, the classification unit 114 may integrate the feature amounts based on a microsaccade into time-series data for each of the measurement conditions and output the time-series data. For example, in the example of the penalty kick described above, the classification unit 114 may integrate the feature amounts corresponding to the four types of measurement conditions “the kicker is imaged small in the video, and the ball flies to the right”, “the kicker is imaged small in the video, and the ball flies to the left”, “the kicker is imaged large in the video, and the ball flies to the right”, and “the kicker is imaged large in the video, and the ball flies to the left” into time-series data for each of the measurement conditions and output the time-series data. In this case, the time-series data of the feature amounts based on a microsaccade is output for four groups. Alternately, the classification unit 114 may integrate the feature amounts based on a microsaccade into a statistical value for each of the measurement conditions (for example, average value of the feature amounts for each of the measurement conditions) and output the statistical value. For example, in the example of rugby or the like described above, the classification unit 114 may average and output, for each of the measurement conditions, the feature amounts corresponding to the four respective measurement conditions of “the opponent imaged small in the video moves to the right”, “the opponent imaged small in the video moves to the left”, “the opponent imaged large in the video moves to the right”, and “the opponent imaged large in the video moves to the left”. In this case, average data of the feature amounts based on a microsaccade is output for four groups. In addition, normalized feature amounts based on a microsaccade may be collectively output to the estimation unit 115 for each of the measurement conditions (step S114).

The feature amounts based on a microsaccade corresponding to each of the measurement conditions are input to the estimation unit 115. The estimation unit 115 evaluates motion performance of the subject 100 on the basis of the feature amounts corresponding to each of the plurality of input measurement conditions, and obtains and outputs an index representing the motion performance of the subject 100. That is, the estimation unit 115 obtains and outputs an index representing the motion performance of the subject 100 on the basis of a difference in feature amounts based on a microsaccade between the subject 100 performing the task according to the movement of the first object and the subject 100 performing the task according to the movement of the second object. However, the sizes of the first object and the second object in the videos viewed from the subject 100 are different from each other. That is, the size of the visual angle formed by the first object and the size of the visual angle formed by the second object in the video in the eye of the subject 100 are different. The first object is, for example, the above-described small kicker 111 or opponent, and the second object is, for example, the above-described large kicker 112 or opponent. As described above, the level of the motion performance of the subject 100 appears as a difference in a feature amount based on a microsaccade of the eye of the subject 100 obtained for each of the first object and the second object having different sizes in the videos. Therefore, the motion performance of the subject 100 can be evaluated on the basis of the difference in the feature amount based on a microsaccade. Hereinafter, a method of evaluating the motion performance of the subject 100 by the estimation unit 115 will be exemplified. For example, the estimation unit 115 evaluates motion performance of the subject 100 on the basis of at least one of the following (A) to (G), and obtains and outputs an index representing the motion performance of the subject 100.

(A) The estimation unit 115 obtains and outputs an index representing that the motion performance of the subject 100 is at a first level when a difference in the feature amount (1) described above (first feature amount: feature amount representing the occurrence frequency of a microsaccade) between the subject 100 performing the task according to the movement of the first object and the subject 100 performing the task according to the movement of the second object is equal to or less than a threshold TH_{A} (first threshold) (for example, in a case where the feature amount is not statistically significant different between the case of the first object and the case of the second object), and obtains and outputs an index representing that the motion performance of the subject 100 is at a second level lower than the first level when the difference in the feature amount (1) (first feature amount) is larger than the threshold TH_{A} (first threshold) (for example, in a case where the feature amount is statistically significantly different between the case of the first object and the case of the second object). The higher the level, the better the motion performance.

(B) The feature amount (1) in (A) may be replaced with the feature amount (3) (first feature amount: feature amount representing the attenuation coefficient of a microsaccade when the eye is modeled by dynamics of the secondary system), and the threshold TH_{A} may be replaced with a threshold TH_{B} (first threshold). In this case, the estimation unit 115 obtains and outputs an index representing that the motion performance of the subject 100 is at the first level when a difference in the feature amount (3) described above (first feature amount: feature amount representing the attenuation coefficient of a microsaccade when the eye is modeled by dynamics of the secondary system) between the subject 100 performing the task according to the movement of the first object and the subject 100 performing the task according to the movement of the second object is equal to or less than the threshold TH_{B} (first threshold), and obtains and outputs an index representing that the motion performance of the subject 100 is at the second level lower than the first level when the difference in the feature amount (3) (first feature amount) is larger than the threshold TH_{B} (first threshold).

(C) The estimation unit 115 obtains and outputs an index representing that the motion performance of the subject 100 is at the first level when a difference in the feature amount (2) described above (second feature amount: feature amount representing the amplitude of a microsaccade) between the subject 100 performing the task according to the movement of the first object and the subject 100 performing the task according to the movement of the second object is equal to or larger than a threshold TH_{C} (second threshold) and a difference in the feature amount (1) (first feature amount: feature amount representing the occurrence frequency of a microsaccade) is equal to or less than the threshold TH_{A} (first threshold), and obtains and outputs an index representing that the motion performance of the subject 100 is at the second level lower than the first level when the difference in the feature amount (2) (second feature amount) is equal to or larger than the threshold TH_{C} (second threshold) and the difference in the feature amount (1) (first feature amount) is larger than the threshold TH_{A} (first threshold).

For example, in a case where the first object is smaller than the second object (in a case where the visual angle formed by the first object in the eye of the subject 100 is smaller than the visual angle formed by the second object), the estimation unit 115 may output an index representing motion performance as follows.
The estimation unit 115 obtains and outputs an index representing that the motion performance of the subject 100 is at the first level when the feature amount (2) of the subject 100 performing the task according to the movement of the second object is larger than the feature amount (2) of the subject 100 when the task according to the movement of the first object is performed by the threshold TH_{C} or more, and the difference between the feature amount (1) of the subject 100 performing the task according to the movement of the second object and the feature amount (1) of the subject 100 when the task according to the movement of the first object is performed is the threshold TH_{A} or less.
The estimation unit 115 obtains and outputs an index representing that the motion performance of the subject 100 is at the second level when the feature amount (2) of the subject 100 performing the task according to the movement of the second object is larger than the feature amount (2) of the subject 100 when the task according to the movement of the first object is performed by the threshold TH_{C} or more, and the difference between the feature amount (1) of the subject 100 performing the task according to the movement of the second object and the feature amount (1) of the subject 100 when the task according to the movement of the first object is performed is larger than the threshold TH_{A}.

(D) The feature amount (2) in (C) may be replaced with the feature amount (4) (second feature amount: feature amount representing the unique angular vibration frequency of a microsaccade when the eye is modeled by dynamics of the secondary system), and the threshold TH_{C} may be replaced with a threshold TH_{D} (second threshold). In this case, the estimation unit 115 obtains and outputs an index representing that the motion performance of the subject 100 is at the first level when a difference in the feature amount (4) described above (second feature amount: feature amount representing the unique angular vibration frequency of a microsaccade when the eye is modeled by dynamics of the secondary system) between the subject 100 performing the task according to the movement of the first object and the subject 100 performing the task according to the movement of the second object is equal to or larger than the threshold TH_{D} (second threshold) and a difference in the feature amount (1) (first feature amount: feature amount representing the occurrence frequency of a microsaccade) is equal to or less than the threshold TH_{A}(first threshold), and obtains and outputs an index representing that the motion performance of the subject 100 is at the second level lower than the first level when the difference in the feature amount (4) is equal to or larger than the threshold TH_{D} (second threshold) and the difference in the feature amount (1) (first feature amount) is larger than the threshold TH_{A} (first threshold).

For example, in a case where the first object in the video is smaller than the second object in the video, the estimation unit 115 may output an index representing motion performance as follows.
The estimation unit 115 obtains and outputs an index representing that the motion performance of the subject 100 is at the first level when the feature amount (4) of the subject 100 performing the task according to the movement of the first object is larger than the feature amount (4) of the subject 100 when the task according to the movement of the second object is performed by the threshold TH_{D} or more, and the difference between the feature amount (1) of the subject 100 performing the task according to the movement of the second object and the feature amount (1) of the subject 100 when the task according to the movement of the first object is performed is the threshold TH_{A} or less.
The estimation unit 115 obtains and outputs an index representing that the motion performance of the subject 100 is at the second level when the feature amount (4) of the subject 100 performing the task according to the movement of the first object is larger than the feature amount (4) of the subject 100 when the task according to the movement of the second object is performed by the threshold TH_{D} or more, and the difference between the feature amount (1) of the subject 100 performing the task according to the movement of the second object and the feature amount (1) of the subject 100 when the task according to the movement of the first object is performed is larger than the threshold TH_{A}.

(E) The feature amount (1) in (C) may be replaced with the feature amount (3) (first feature amount: feature amount representing the attenuation coefficient of a microsaccade when the eye is modeled by dynamics of the secondary system), and the threshold TH_{A} may be replaced with the threshold TH_{B} (first threshold).

(F) The feature amount (1) in (D) may be replaced with the feature amount (3) (first feature amount: feature amount representing the attenuation coefficient of a microsaccade when the eye is modeled by dynamics of the secondary system), and the threshold TH_{A} may be replaced with the threshold TH_{B} (first threshold).

(G) The estimation unit 115 may obtain an index representing that the motion performance of the subject 100 is at the first level when a ratio of a difference in the first feature amount (feature amount of (1) or (3) described above) to a difference in the second feature amount (feature amount of (2) or (4) described above) between the subject 100 performing a task according to the movement of the first object and the subject 100 performing a task according to the movement of the second object (the ratio is difference in the first feature amount/difference in the second feature amount) is equal to or less than a threshold TH_{G} (third threshold), and obtain and output an index representing that the motion performance of the subject 100 is at the second level lower than the first level when the ratio is larger than the threshold TH_{G} (third threshold).

Note that, for example, the estimation unit 115 performs binary determination of whether the motion performance of the subject 100 is high or low, and outputs an index representing that the motion performance of the subject 100 is at the first level of high motion performance or an index representing that the motion performance of the subject 100 is at the second level of low motion performance. Alternatively, for example, the estimation unit 115 may obtain and output an index representing a level representing the level of the motion performance of the subject 100 among levels of three or more values each representing the level of the motion performance of the subject 100. In this case, threshold determination (A) to (G) described above may be performed so that the level of the motion performance of the subject 100 can be divided into N or more levels (where N is an integer of three or more).

As described above, the motion performance according to the surrounding situation during motion of the subject 100 can be appropriately evaluated.

### Second Embodiment

A second embodiment will be described. In the first embodiment, the motion performance of the subject 100 is estimated from a feature amount based on a microsaccade of the eye of the subject 100 performing the task according to the movement of the object in the video presented from the video presentation unit 12. However, an object in a real space may be used instead of the object in a video. In the second embodiment, an example in which the motion performance of the subject 100 is estimated from a feature amount based on a microsaccade of the eye of the subject 100 performing a task according to movement of an object in an actual sports environment will be described. Hereinafter, differences from the first embodiment will be mainly described, and description of matters already described will be simplified using the same reference numerals.

### <Configuration>

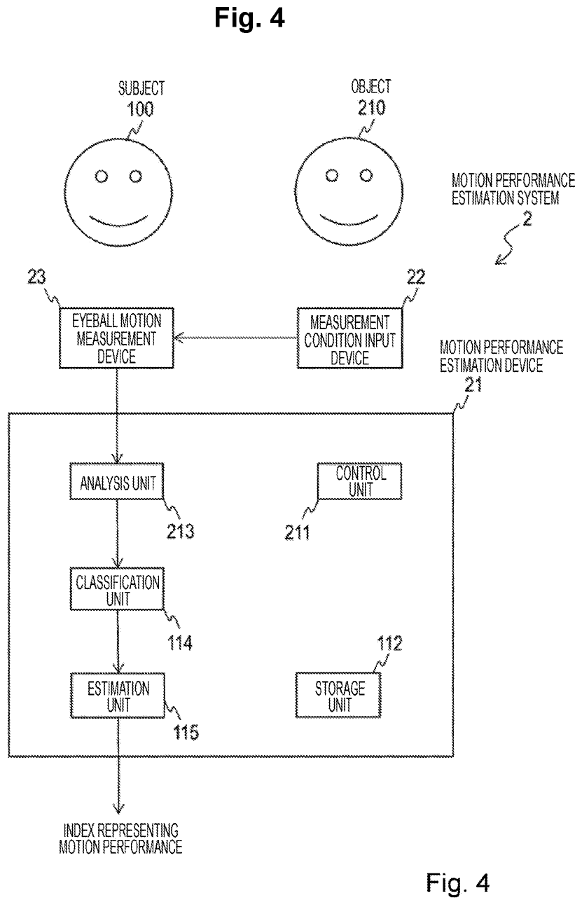

As illustrated in FIG. 4, a motion performance estimation system 2 of the present embodiment includes a motion performance estimation device 21 that estimates motion performance of a subject 100, a measurement condition input device 22 that inputs a measurement condition of an object 210 in a real space, and an eyeball motion measurement device 23 that measures eyeball motion of the subject 100. The motion performance estimation device 21 includes a control unit 211, a storage unit 112, an analysis unit 213, a classification unit 114, and an estimation unit 115. The motion performance estimation device 21 performs each type of processing on the basis of the control unit 211, and data obtained in each processing is stored in the storage unit 112 one by one, and read and used as necessary. The eyeball motion measurement device 23 is a device such as an eye tracker that measures eyeball motion and the visual field of the subject 100.

### <Processing>

The motion performance estimation device 21 obtains and outputs an index representing the motion performance of the subject 100 based on a difference in a feature amount based on a microsaccade between the subject 100 performing a task according to movement of a first object and the subject 100 performing a task according to movement of a second object. The size of the visual angle formed by the first object and the size of the visual angle formed by the second object in the eye of the subject 100 are different. However, the first object and the second object of the present embodiment are the object 210 in the real space. Hereinafter, an example of this processing will be described.

The measurement condition input unit 22 is a device that inputs information of a first measurement condition for the subject 100 and the object 210 to the eyeball motion measurement device 13. The first measurement condition is a condition representing a result according to movement of the object 210 corresponding to the task that the subject 100 is caused to be performed. For example, in a case where the subject 100 is caused to perform a task of predicting whether the ball kicked by the kicker (object 210) flies to the right or the left in the scene of the penalty kick described above, first measurement conditions are “the ball flies to the right” and “the ball flies to the left”. Furthermore, for example, in a case where the subject 100 is caused to perform a task of predicting a movement direction of an opponent (object 210) who approaches holding a ball in a scene such as rugby, first measurement conditions are “the opponent moves to the right” and “the opponent moves to the left”. For example, in a scene where the subject 100 performs a task according to the movement of the object 210, the measurement condition input unit 22 automatically selects a first measurement condition at each time or in each time section in real time according to the position and movement of the object 210 in the real space, and inputs the selected first measurement condition to the eyeball motion measurement device 13. Alternatively, the object 210 or a person other than the object 210 observing the state of the object 210 may select a first measurement condition at each time or in each time section in real time, and input information indicating the selected first measurement condition to the measurement condition input device 22, and the measurement condition input device 22 may input the first measurement condition to the measurement condition input device 22 (step S22).

The eyeball motion measurement device 23 receives a first measurement condition at each time or in each time section. The eyeball motion measurement device 23 acquires eyeball movement of the subject 100 viewing the object 210 in the real space (for example, position of the eyeball at each time) and the visual field including the object 210 viewed by the subject 100. The eyeball motion measurement device 23 outputs the acquired information of the eyeball motion and the visual field to the analysis unit 213 in association with the first measurement condition (step S213).

The information of the eyeball motion and the visual field of the subject 100 and the first measurement condition associated with the eyeball motion and the visual field are input to the analysis unit 213. The analysis unit 213 obtains a second measurement condition representing the size of the object 210 viewed by the subject 100 from the input visual field information of the subject 100. The size of the object 210 viewed by the subject 100 is the size of the object 210 perceived by the subject 100, and corresponds to the size of the image of the object 210 reflected in the retina of the eye of the subject 100. For example, in a case where the subject 100 is caused to perform a task of predicting whether the ball kicked by the kicker (object 210) flies to the right or the left in the scene of the penalty kick described above, second measurement conditions are “the kicker (object 210) looks far from the subject 100 and small” and “the kicker (object 210) looks close to the subject 100 and large”. Furthermore, for example, in a case where the subject 100 is caused to perform a task of predicting a movement direction of an opponent (object 210) who approaches holding a ball in a scene such as rugby, second measurement conditions are “the opponent (object 210) looks far from the subject 100 and small” and “the opponent (object 210) looks close to the subject 100 and large”. With such second measurement conditions and the above-described first measurement conditions, information equivalent to the measurement conditions described in the first embodiment can be obtained. Hereinafter, a set of a first measurement condition and a second measurement condition will be referred to as a measurement condition. The analysis unit 213 extracts a feature amount based on a microsaccade of the eye of the subject 100 from the input measurement result of the eyeball motion. The analysis unit 213 outputs the extracted feature amount based on a microsaccade and the measurement condition corresponding to the measurement result of the eyeball motion that is the basis of the feature amount to the classification unit 114 in association with each other. The processing is similar to that of the first embodiment (step S213).

Processing of steps S22, S13, and S213 described above is performed a plurality of times. As a result, at least feature amounts based on a plurality of microsaccades of the subject 100 are obtained for objects 210 having different sizes as viewed from the subject 100. Thereafter, the classification unit 114 performs processing of step S114 described above, and the estimation unit 115 performs processing of step S115 described above, and obtains and outputs an index representing the motion performance of the subject 100.

As described above, the motion performance according to the surrounding situation during motion of the subject 100 can be appropriately evaluated.

### [Hardware Configuration]

The motion performance estimation device 11, 21 according to each embodiment is a device formed with a general-purpose or dedicated computer executing a predetermined program, the computer including a processor (a hardware processor) such as a central processing unit (CPU) and a memory such as a random access memory (RAM) and a read only memory (ROM), for example. That is, the motion performance estimation device 11, 21 according to each embodiment include processing circuitry designed to implement each component included in each motion performance estimation device. The computer may include one processor and one memory, or may include a plurality of processors and a plurality of memories. The program may be installed in the computer, or may be recorded in a ROM or the like in advance. Also, some or all of the processing units may be formed with an electronic circuit that executes the processing functions, rather than an electronic circuit (circuitry) that forms the functional components by reading the program like a CPU. Also, an electronic circuit forming one device may include a plurality of CPUs.

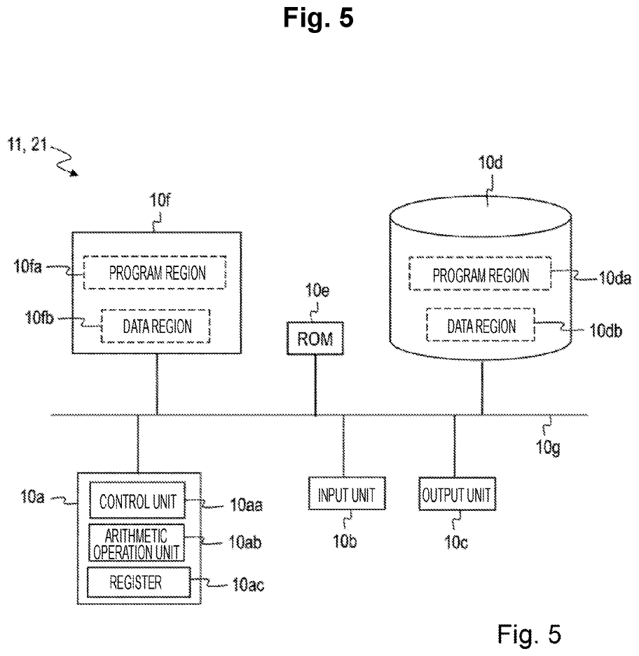

FIG. 5 is a block diagram illustrating a hardware configuration of the motion performance estimation device 11, 21 according to each embodiment. As illustrated as the example in FIG. 5, the motion performance estimation device 11, 21 in this example includes a central processing unit (CPU) 10a, an input unit 10b, an output unit 10c, a random access memory (RAM) 10d, a read only memory (ROM) 10e, an auxiliary storage device 10f, and a bus 10g. The CPU 10a in this example includes a control unit 10aa, an arithmetic operation unit 10ab, and a register 10ac, and performs various arithmetic operations in accordance with various programs read into the register 10ac. Meanwhile, the input unit 10b is an input terminal to which data is input, a keyboard, a mouse, a touch panel, or the like. Also, the output unit 10c is an output terminal from which data is output, a display, a LAN card or the like controlled by the CPU 10a that has read a predetermined program. Further, the RAM 10d is a static random access memory (SRAM), a dynamic random access memory (DRAM), or the like, and incudes a program region 10da in which a predetermined program is stored and a data region 10db in which various kinds of data are stored. Further, the auxiliary storage device 10f is a hard disk, a magneto-optical disc (MO), a semiconductor memory, or the like, for example, and includes a program region 10fa in which a predetermined program is stored and a data region 10fb in which various kinds of data are stored. Meanwhile, the bus 10g connects the CPU 10a, the input unit 10b, the output unit 10c, the RAM 10d, the ROM 10e, and the auxiliary storage device 10f so that information can be exchanged among these components. The CPU 10a writes, into the program region 10da of the RAM 10d, the program stored in the program region 10fa of the auxiliary storage device 10f, in accordance with a read operating system (OS) program. Likewise, the CPU 10a writes, into the data region 10db of the RAM 10d, the various kinds of data stored in the data region 10fb of the auxiliary storage device 10f. The addresses in the RAM 10d at which the program and the data are written are stored into the register 10ac of the CPU 10a. The control unit 10aa of the CPU 10a sequentially reads these addresses stored in the register 10ac, reads the program and the data from the regions in the RAM 10d indicated by the read addresses, causes the arithmetic operation unit 10ab to sequentially execute arithmetic operations indicated by the program, and stores results of the arithmetic operations into the register 10ac. With such a configuration, the functional components of the motion performance estimation device 11, 21 are obtained.

The program mentioned above can be recorded in a computer-readable recording medium. The computer-readable recording medium in an example is a non-transitory recording medium. Examples of such a recording medium include a magnetic recording device, an optical disc, a magneto-optical recording medium, and a semiconductor memory.

The program is distributed by selling, giving, or renting portable recording media such as DVDs or CD-ROMs recording the program thereon. Furthermore, a configuration in which the program is stored in a storage device in a server computer and the program is distributed by transferring the program from the server computer to other computers via a network may also be employed. As described above, the computer executing such a program first stores the program recorded in the portable recording medium or the program transferred from the server computer temporarily into a storage device of the computer, for example. The computer then reads the program stored in the storage device, and performs processing in accordance with the read program at the time of execution of the processing. Also, in other execution modes of the program, the computer may read the program directly from the portable recording medium and performs processing in accordance with the program, or alternatively, the computer may sequentially execute processing in accordance with the received program every time the program is transferred from the server computer to the computer. Alternatively, the above processing may be executed by a so-called application service provider (ASP) service that implements a processing function only by issuing an instruction to execute the program and acquiring the result, without transferring the program from the server computer to the computer. Note that the program according to the present embodiment includes information used for processing by an electronic computer and equivalent to the program (data, or the like, that is not a direct command to the computer but has property that defines processing of the computer).

Although this device is formed with a computer executing a predetermined program in each embodiment, at least some of the processing contents may be implemented by hardware.

### Modification Examples and the Like

Note that the present invention is not limited to the embodiments described above. For example, in the embodiments described above, an example of evaluating motion performance in playing soccer or rugby has been described. However, this does not limit the invention and the invention may be applied in a case of evaluating performance in motion that requires a response in response to movement of an object, such as baseball, football, tennis, badminton, boxing, kendo, fencing, etc. Furthermore, the object may be an entire human, a part of a human such as an arm, or a thing such as a ball. Furthermore, a first feature amount may be a function value for the occurrence frequency and the unique angular vibration frequency of a microsaccade described above, or a second feature amount may be a function value for the amplitude and the unique angular vibration frequency of a microsaccade described above. In the first embodiment, regardless of whether a first object is presented or a second object is presented (that is, regardless of the measurement condition), a distance between a subject 100 and the video presentation unit 12 that presents a video including the first object or the second object is constant or substantially constant. However, the distance between the video presentation unit 12 and the subject 100 may change. However, in this case, regardless of the distance between the video presentation unit 12 and the subject 100, the sizes of videos presented from the video presentation unit 12 need to be adjusted such that the size of a first object image in a video appearing in the retina of the eye of the subject 100 is constant or substantially constant, the size of a second object image in a video appearing in the retina of the eye of the subject 100 is constant or substantially constant, and the size of the first object image and the size of the second object image in the video appearing in the retina of the eye of the subject 100 are different from each other.

Also, various kinds of processing described above may be performed not only in a chronological manner in accordance with the description but also in parallel or individually in accordance with the processing ability of the devices that perform the processing or as needed. It is needless to say that appropriate modifications can be made without departing from the gist of the present invention.

### REFERENCE SIGNS LIST

1, 2 Motion performance estimation device
115 Estimation unit

1. A motion performance estimation device comprising a processor configured to execute operations comprising:
obtaining and outputting an index, the index representing motion performance of a subject on a basis of a difference between a first feature amount of a microsaccade of the subject when performing a first task according to movement of a first object and a second feature amount of the microsaccade of the subject when performing a second task according to movement of a second object,
wherein a first magnitude of a first visual angle formed by the first object in an eye of the subject is distinct from a second magnitude of a second visual angle formed by the second object in the eye of the subject.

2. The motion performance estimation device according to claim 1,
wherein a first feature amount based on the microsaccade includes a third feature amount representing an occurrence frequency or an attenuation coefficient of the microsaccade.

3. The motion performance estimation device according to claim 2,
wherein the obtaining and outputting further comprise obtaining the index, the index represents that motion performance of the subject is at a first level when a difference in the third feature amount between the subject performing the first task according to movement of the first object and the subject performing the second task according to movement of the second object is equal to or less than a first threshold, and
the obtaining and outputting further comprise obtaining the index, and the index represents that motion performance of the subject is at a second level that is lower than the first level when a difference in the third feature amount is larger than the first threshold.

4. The motion performance estimation device according to claim 1,
wherein the first feature amount based on the microsaccade includes a third feature amount representing an occurrence frequency or an attenuation coefficient of the microsaccade, and a fourth feature amount representing an amplitude or a unique angular vibration frequency of the microsaccade.

5. The motion performance estimation device according to claim 4,
wherein the obtaining and outputting further comprises:
obtaining the index, the index represents that motion performance of the subject is at a first level when a difference in the fourth feature amount between the subject performing the first task according to movement of the first object and the subject performing the second task according to movement of the second object is equal to or more than a second threshold and a difference in the third feature amount is equal to or less than a first threshold, and
obtaining the index, the index represents that motion performance of the subject is at a second level that is lower than the first level when a difference of the fourth feature amount is equal to or more than the second threshold and a difference in the third feature amount is larger than the first threshold.

6. The motion performance estimation device according to claim 4,
wherein the obtaining and outputting further comprises:
obtaining the index, the index represents that motion performance of the subject is at a first level when a ratio of a difference in the third feature amount to a difference in the fourth feature amount between the subject performing the first task according to movement of the first object and the subject performing the second task according to movement of the second object is equal to or less than a third threshold, and
obtaining the index, wherein the index represents that motion performance of the subject is at a second level lower than the first level when the ratio is larger than the third threshold.

7. A motion performance estimation method, comprising:
obtaining and outputting an index, the index representing motion performance of a subject on a basis of a difference between a first feature amount of a microsaccade of the subject when performing a first task according to movement of a first object and a second feature amount of the microsaccade of the subject when performing a second task according to movement of a second object,
wherein a first magnitude of a first visual angle formed by the first object in an eye of the subject is distinct from a second magnitude of a second visual angle formed by the second object in the eye of the subject.

8. A computer-readable non-transitory recording medium storing a computer-executable program instructions that when executed by a processor cause a computer system to execute operations comprising:
obtaining and outputting an index, the index representing motion performance of a subject on a basis of a difference between a first feature amount of a microsaccade of the subject when performing a first task according to movement of a first object and a second feature amount of the microsaccade of the subject when performing a second task according to movement of a second object,
wherein a first magnitude of a first visual angle formed by the first object in an eye of the subject is distinct from a second magnitude of a second visual angle formed by the second object in the eye of the subject.

9. The motion performance estimation device according to claim 1, wherein the difference between the first feature amount and the second feature amount indicates a size of an attention range of the subject, and the size of the attention range of the subject correlates with a reaction speed of the subject.

10. The motion performance estimation method according to claim 7, wherein a first feature amount based on the microsaccade includes a third feature amount representing an occurrence frequency or an attenuation coefficient of the microsaccade.

11. The motion performance estimation method according to claim 10,
wherein the obtaining and outputting further comprises obtaining the index, the index represents that motion performance of the subject is at a first level when a difference in the third feature amount between the subject performing the first task according to movement of the first object and the subject performing the second task according to movement of the second object is equal to or less than a first threshold, and
the obtaining and outputting further comprise obtaining the index, and the index represents that motion performance of the subject is at a second level that is lower than the first level when a difference in the third feature amount is larger than the first threshold.

12. The motion performance estimation method according to claim 7,
wherein the first feature amount based on the microsaccade includes a third feature amount representing an occurrence frequency or an attenuation coefficient of the microsaccade, and a fourth feature amount representing an amplitude or a unique angular vibration frequency of the microsaccade.

13. The motion performance estimation method according to claim 12, wherein the obtaining and outputting further comprises:
obtaining the index, the index represents that motion performance of the subject is at a first level when a difference in the fourth feature amount between the subject performing the first task according to movement of the first object and the subject performing the second task according to movement of the second object is equal to or more than a second threshold and a difference in the third feature amount is equal to or less than a first threshold, and
obtaining the index, the index represents that motion performance of the subject is at a second level that is lower than the first level when a difference of the fourth feature amount is equal to or more than the second threshold and a difference in the third feature amount is larger than the first threshold.

14. The motion performance estimation method according to claim 12, wherein the obtaining and outputting further comprises:
obtaining the index, the index represents that motion performance of the subject is at a first level when a ratio of a difference in the third feature amount to a difference in the fourth feature amount between the subject performing the first task according to movement of the first object and the subject performing the second task according to movement of the second object is equal to or less than a third threshold, and
obtaining the index, wherein the index represents that motion performance of the subject is at a second level lower than the first level when the ratio is larger than the third threshold.

15. The motion performance estimation method according to claim 7, wherein the difference between the first feature amount and the second feature amount indicates a size of an attention range of the subject, and the size of the attention range of the subject correlates with a reaction speed of the subject.

16. The computer-readable non-transitory recording medium according to claim 8, wherein a first feature amount based on the microsaccade includes a third feature amount representing an occurrence frequency or an attenuation coefficient of the microsaccade.

17. The computer-readable non-transitory recording medium according to claim 16, wherein the obtaining and outputting further comprises obtaining the index, the index represents that motion performance of the subject is at a first level when a difference in the third feature amount between the subject performing the first task according to movement of the first object and the subject performing the second task according to movement of the second object is equal to or less than a first threshold, and
the obtaining and outputting further comprise obtaining the index, and the index represents that motion performance of the subject is at a second level that is lower than the first level when a difference in the third feature amount is larger than the first threshold.

18. The computer-readable non-transitory recording medium according to claim 8, wherein the first feature amount based on the microsaccade includes a third feature amount representing an occurrence frequency or an attenuation coefficient of the microsaccade, and a fourth feature amount representing an amplitude or a unique angular vibration frequency of the microsaccade.

19. The computer-readable non-transitory recording medium according to claim 18, wherein the obtaining and outputting further comprises:
obtaining the index, the index represents that motion performance of the subject is at a first level when a difference in the fourth feature amount between the subject performing the first task according to movement of the first object and the subject performing the second task according to movement of the second object is equal to or more than a second threshold and a difference in the third feature amount is equal to or less than a first threshold, and
obtaining the index, the index represents that motion performance of the subject is at a second level that is lower than the first level when a difference of the fourth feature amount is equal to or more than the second threshold and a difference in the third feature amount is larger than the first threshold.

20. The computer-readable non-transitory recording medium according to claim 18, wherein the obtaining and outputting further comprises:
obtaining the index, the index represents that motion performance of the subject is at a first level when a ratio of a difference in the third feature amount to a difference in the fourth feature amount between the subject performing the first task according to movement of the first object and the subject performing the second task according to movement of the second object is equal to or less than a third threshold, and
obtaining the index, wherein the index represents that motion performance of the subject is at a second level lower than the first level when the ratio is larger than the third threshold.
